(19) United States
(12) Patent Application Publication
(10) Pub. No.: US 2016/0109423 A1
(43) Pub. Date: Apr. 21, 2016

(54) MEASURING DEVICE FOR MEASURING A SURFACE LAYER ON AN OBJECT TO BE MEASURED, PARTICULARLY ON A FOODSTUFF

(71) Applicant: FRESHDETECT GMBH, Karlsfeld (DE)

(72) Inventors: Mathias REICHL, Kelheim (DE); Rolf-Dieter KLEIN, Muenchen (DE)

(73) Assignee: FRESHDETECT GMBH, Karlsfeld (DE)

(21) Appl. No.: 14/888,543

(22) PCT Filed: May 7, 2014
(86) PCT No.: PCT/EP2014/001230

§ 371 Date: Nov. 2, 2015

(30) Foreign Application Priority Data
May 8, 2013 (DE) 10 2013 008 003.7

(51) Int. Cl. G01N 33/12; G01N 21/94; G01N 21/64

(52) U.S. Cl. CPC G01N 33/12; G01N 21/6486; G01N 21/94; G01N 2201/0612; G01N 2201/0621

(57) ABSTRACT

The invention relates to a measuring device (1; 30) for measuring a surface coating (18) on an object to be measured (19; 24) such as a foodstuff, particularly for the measurement of metabolic products from bacteria on meat intended for consumption, said measuring device having at least one excitation source (3-6) for the excitation of luminescence in the surface coating (18) on the object to be measured (19; 24), such that the surface coating (18) emits a luminescent radiation and having at least one optical sensor (10) for detecting the luminescent radiation emitted by the surface coating (18). According to the invention, the measuring device (1; 30) measures the luminescent radiation from the surface coating (18) at a plurality of measuring points spaced at a distance from one another (M1-M4) on the foodstuff to be monitored (19; 24), particularly at four measuring points (M1-M4).

The invention relates to a measuring device for measuring a surface layer on an object to be measured (e.g. foodstuff), in particular for measuring metabolites of bacteria on meat that is intended for consumption.

Such a measuring device is known by the name “Fresh Scan” from a research project. This known measuring device is based on the finding that meat, during storage, becomes increasingly populated with bacteria whose metabolites (e.g. porphyrins) fluoresce, so that the measurement of the fluorescent radiation emitted by the metabolites of the bacteria allows a conclusion to be drawn about the freshness and the total bacterial count (TBC) of the meat.

For exciting the fluorescent radiation, the known measuring device has a laser, the radiation of which is directed at the meat to be tested so that the metabolites of the bacteria on the meat emit fluorescent radiation, which is then detected by an optical sensor, the optical sensor scanning specific wavelengths in the fluorescent radiation that are characteristic of the fluorescent radiation of the metabolites of the bacteria.

On the one hand, the known measuring device accordingly does not take into consideration the whole spectrum of the fluorescent radiation, but only specific characteristic wavelengths of the fluorescent radiation.

On the other hand, it should be mentioned that the known measuring device measures the fluorescent radiation only at a single measuring point.

Incorrect measurements therefore occasionally occur during operation of the known measuring device described above.

In respect of the prior art, reference is further to be made to DE 10 2011 100 507 A1, DE 27 28 717 A1, U.S. Pat. No. 6,597,932 B2, WO 97/08538 A1, WO 99/57529 A1, WO 2007/079943 A1, EP 0 128 889 B2, DE 602 08 823 T2, U.S. Pat. No. 5,760,406 A, U.S. Pat. No. 5,914,247 A, AT 414 275 B, DE 103 15 541 A1, DE 10 2005 051 643 A1, WO 2003/070080 A1, U.S. Pat. No. 5,658,798 A1, U.S. Pat. No. 5,474,910 A, EP 1 329 514 A2, U.S. Pat. No. 4,866,283 A, DE 44 20 401 A1 and U.S. Pat. No. 6,649,412 B1. However, these specifications merely disclose measuring devices or measuring principles which are not optimal.

Accordingly, the object underlying the invention is to improve the known measuring device described at the beginning.

The object is achieved by a measuring device according to the invention according to the main claim.

The measuring device according to the invention has, in conformity with the prior art, at least one excitation source for exciting luminescence in the surface layer of the foodstuff to be monitored so that the surface layer emits luminescent radiation.

In a preferred embodiment of the invention, the excitation source—as in the known measuring device—is a light source, so that the luminescent radiation excited by the light source is photoluminescent radiation, in particular fluorescent radiation or phosphorescent radiation. However, the invention is not limited in respect of the excitation of the luminescent radiation to optical excitation. Rather, it is also conceivable in principle within the scope of the invention to use other types of luminescence, such as, for example, electroluminescence, cathodoluminescence, chemoluminescence, bioluminescence, triboluminescence, thermoluminescence, sonoluminescence, radioluminescence, ionoluminescence or piezoluminescence. The decay time of the fluorescence can hereby also be evaluated.

In the preferred embodiment of the invention having a light source for exciting photoluminescence, the light source preferably emits a spectrum that has a wavelength range of from 350 nm to 550 nm. In a preferred embodiment, the light source emits a wavelength of 405 nm.

For example, the light source can be a laser diode or a light-emitting diode, but it is in principle also possible to use a different light source, such as, for example, a filament lamp.

The measuring device additionally has, in conformity with the known measuring device described at the beginning, at least one optical sensor for measuring the luminescent radiation that is emitted by the surface layer on the foodstuff.

In the preferred embodiment, the optical sensor measures the luminescent radiation from all the measuring points, it being possible for the measurement at the individual measuring points to take place, for example, sequentially in terms of time.

Alternatively, however, it is also possible for each measuring point to have its own associated optical sensor which measures the luminescent radiation at that particular measuring point.

When a single optical sensor is used for the measurement at all the measuring points, the optical sensor preferably has a sufficiently large measuring angle that all the measuring points lie within the measuring angle, so that the optical sensor is able to measure the luminescent radiation from all the measuring points.

For example, the luminescent radiation emitted by the surface layer can be guided to the optical sensor via a light guide having a suitable numerical aperture (NA).

It should further be mentioned that the optical sensor is preferably a spectral photometer which measures a wavelength spectrum of the luminescent radiation of the surface layer. The measuring device according to the invention thereby differs from the known measuring device described at the beginning, in which only the intensity of the luminescent radiation at specific characteristic wavelengths is measured, whereas the spectral photometer detects the entire wavelength spectrum of the luminescent radiation. This is advantageous because characteristic wavelength spectra of the luminescent radiation can thereby be detected, as a result of which incorrect measurements can largely be avoided. Within the scope of the invention, therefore, there is also the possibility of signal evaluation by so-called “spectral imaging”, which is known per se from the prior art.

It should further be mentioned that a plurality of measuring points is preferably provided, the individual measuring points preferably being distributed around the optical axis of the optical sensor. This is advantageous because the optical sensor can then more easily detect the luminescent radiation from all the measuring points.

In the preferred embodiment of the invention, the measuring device takes into consideration not only the wavelength spectrum of the luminescent radiation that is emitted by the surface layer but also the surface temperature of the surface layer on the foodstuff that is to be monitored. To that end, the measuring device according to the invention preferably has a pyrometer, which permits contactless temperature measurement.

The measuring device according to the invention preferably further comprises a colorimeter for measuring the color of the surface layer without excitation of luminescence, such colorimeters being known per se from the prior art and therefore not requiring further description.

Within the scope of the invention, the measuring accuracy can further be improved if the decay behavior over time of the luminescent radiation of the surface layer is also taken into consideration. The measuring device according to the invention therefore preferably has a measuring element which measures the decay behavior over time of the luminescent radiation.

In addition, the measuring device according to the invention can also have a pH meter for measuring the pH value of the surface layer on the foodstuff.

The measuring device according to the invention can further have a resistance measuring device for measuring the electrical surface resistance of the surface layer and/or of the foodstuff, as a result of which the measuring accuracy can be improved further.

It should further be mentioned that the measuring device according to the invention comprises an evaluation unit for qualifying and/or for quantifying the surface layer and/or the foodstuff and for generating a corresponding output signal. The output signal of the evaluation unit can indicate, for example, one of the following properties of the foodstuff to be tested:
Bacterial count of metabolites of bacteria in the surface layer, in particular of porphyrins, in particular of protoporphyrin IX.
Type of foodstuff from the group meat, fish, vegetable and/or fruit.
Type of meat from the group pork, beef, poultry, lamb, venison, horsemeat and/or dog meat.
Edibility of the foodstuff in dependence on the freshness of the foodstuff.

It has already been pointed out above that meat, during storage, becomes increasingly populated with bacteria whose metabolites emit a characteristic fluorescent radiation, so that measurement of the fluorescent radiation allows a conclusion to be drawn about the freshness of the meat. When evaluating the measured fluorescent radiation, only the characteristic wavelength spectrum that is emitted by the bacterial metabolites in question (e.g. porphyrins) is selectively to be taken into consideration, where possible. The evaluation unit therefore measures at the peaks of the luminescent radiation the wavelength of each peak and the intensity of each peak. The evaluation unit then preferably calculates the intensity ratio of the peaks, that is to say, for example, the ratio of the intensity of the first peak and the intensity of the second peak. The evaluation unit preferably further determines the wavelength correspondence between, on the one hand, the wavelengths of the measured peaks of the luminescent radiation and, on the other hand, given characteristic wavelengths which are characteristic for the fluorescent radiation of the bacterial metabolites. For example, the characteristic wavelengths can be the wavelengths of peaks in the fluorescent radiation of porphyrins, in particular of protoporphyrin IX. The intensity ratio of the measured peaks and the wavelength correspondence of the measured peaks with the characteristic wavelengths then allow an assessment to be made of whether the measured radiation actually originates from bacterial metabolites or is based on faults.

The evaluation unit can further evaluate the signal shape of the peaks in order to assess whether the detected fluorescent radiation is generated by the surface layer or by faults.

The evaluation unit can then qualify and/or quantify the surface layer on the foodstuff (e.g. meat) in dependence on at least one of the following parameters, a corresponding output signal then being generated:
total value, which reflects the intensities of the luminescent radiation at all the measuring points, for example mean of the measured intensities at the individual measuring points,
intensities at the individual measuring points,
surface temperature of the foodstuff,
color of the surface layer on the foodstuff,
decay behavior over time of the luminescent radiation emitted by the surface layer on the foodstuff,
intensity ratio of the peaks of the luminescent radiation,
wavelength correspondence between the peaks of the luminescent radiation and the given characteristic wavelengths,
pH value,
surface resistance.

In the preferred embodiment of the invention, the above-mentioned input parameters are evaluated by a fuzzy logic, which is known per se from the prior art and therefore does not require further description.

It should further be mentioned that the measuring device according to the invention is preferably portable, for example in the form of a hand-held device. This allows it to be used, for example, in gastronomy or in meat processing plants.

The measuring device according to the invention can be supplied with power by an integrated battery, for example, the battery preferably being a rechargeable battery.

In addition, the measuring device according to the invention preferably has a display in order to allow the output signal of the evaluation unit or other operating parameters of the measuring device to be displayed. For example, the display can be an LCD display (LCD: liquid crystal display).

It is also important that the measuring device according to the invention permits a rapid measurement, so that the measuring device can also be used in production lines in meat processing plants without the processing speed being impaired. The measuring time is therefore preferably less than 10 seconds, 1 second or 50 milliseconds.

In addition, the measuring device according to the invention preferably has a data interface for configuring the measuring device and/or for emitting measured data. For example, the data interface can be a USB interface (USB: universal serial bus), a Bluetooth interface, a WLAN interface (WLAN: wireless local area network) and/or an RFID interface (RFID: radio-frequency identification).

In the preferred embodiment of the invention, the measuring device has a transparent and removable cap, excitation of the luminescent radiation and measurement of the luminescent radiation taking place through the cap. For example, the cap can simply be fitted to and removed from a measuring head of the measuring device.

It should further be mentioned that each measuring point preferably has its own associated excitation source, so that the excitation of luminescence at each measuring point takes place by the excitation source associated with that measuring point. However, it is also possible, as an alternative, that only a single excitation source is provided, which effects the excitation of luminescence at all the measuring points.

It is also conceivable within the scope of the invention to combine the above-described fluorescence evaluation with Raman spectroscopy known per se. A single light source can thereby be used both for fluorescence excitation and for Raman spectroscopy. The excitation is then preferably carried out by a laser in the green wavelength range having a wavelength in the range of from 510 nm to 550 nm. A single spectrometer is then also sufficient for the evaluation, because the Stokes lines evaluated within the context of Raman spectroscopy overlap with the fluorescence response. This would be a very cost-effective dual measuring method with which, using only a single measuring device, the meat quality could be verified and the type of meat (horsemeat, pork, etc.) could be checked.

The invention is also based on the finding that the disruptive incorrect measurements in the case of the measuring device mentioned at the beginning can be caused as a result of the fact that the measurement takes place at a locally limited irregularity on the surface of the meat, for example in the region of a fat streak or of a bone.

The invention therefore also includes the general technical teaching of carrying out the measurement not at only a single measuring point on the foodstuff to be monitored but at a plurality of measuring points, the measuring points being spaced apart from one another. For example, the measuring device according to the invention can have four different measuring points, but a larger number (e.g. 5, 6, 7, 8 or n>8) or a smaller number (e.g. 2, 3) of measuring points is also possible within the scope of the invention.

Finally, the invention also includes the novel use of such a measuring device for measuring metabolites of bacteria on a foodstuff that is intended for consumption, in particular on meat.

The measuring device according to the invention can also be used on a production line on which foodstuffs are processed, the foodstuff (e.g. meat) being transported along the production line and there being subjected to various processing steps (e.g. reception, cutting, portioning, weighing, measuring, preparation, plating up and/or packaging). The freshness of the meat or foodstuff is thus preferably also measured along the production line by means of the measuring device according to the invention. Where the foodstuff is packaged using transparent wrapping film, the freshness can also be measured through the packaging.

It should further be mentioned that the invention is not limited to the measurement of meat. Rather, the principle according to the invention is also suitable for measuring surface layers on other types of foodstuffs, such as, for example, fish, fruit and vegetables.

Furthermore, the object to be measured does not have to be a foodstuff. Rather, the measuring device according to the invention is also suitable for measuring other objects to be measured, such as, for example, the body surface of a living human being in order, for example, to be able to assess injuries. The measuring device according to the invention can accordingly also be in the form of, for example, an wound scanner.

Other advantageous further developments are characterized in the dependent claims or will be described in greater detail below by means of the figures, together with the description of preferred embodiments of the invention. In the figures:

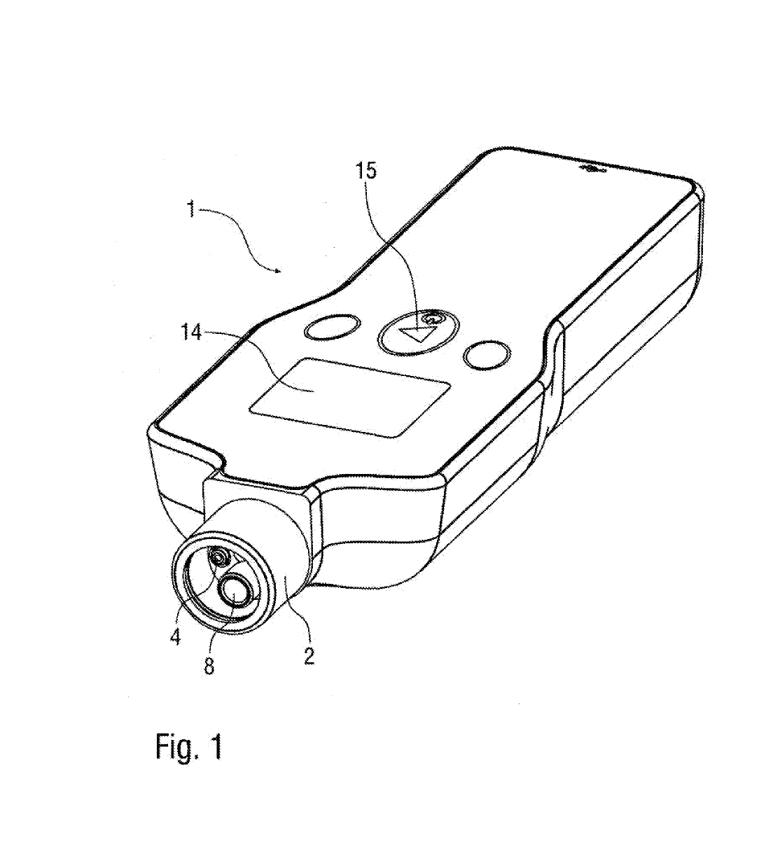

FIG. 1 is an oblique perspective front view of a measuring device according to the invention,

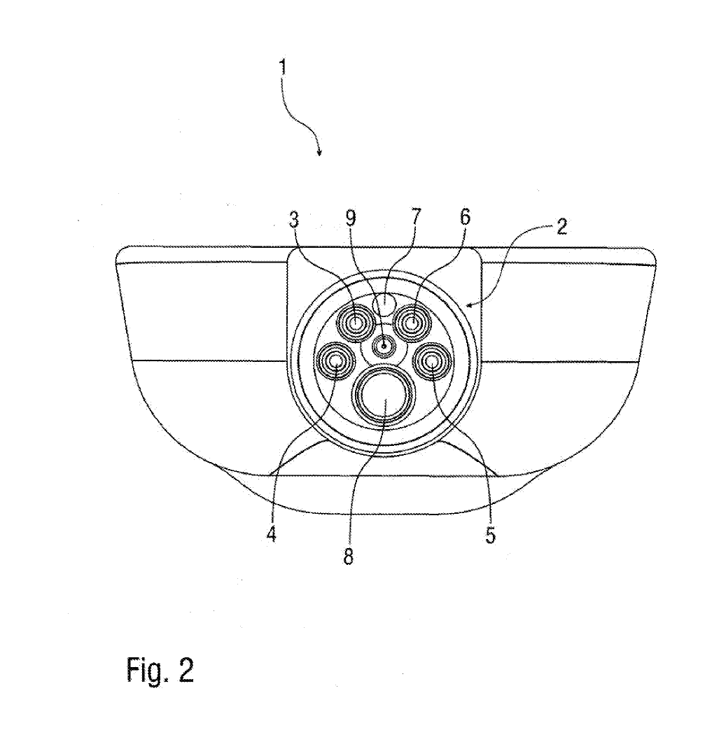

FIG. 2 is a front view of the measuring head of the measuring device according to FIG. 1,

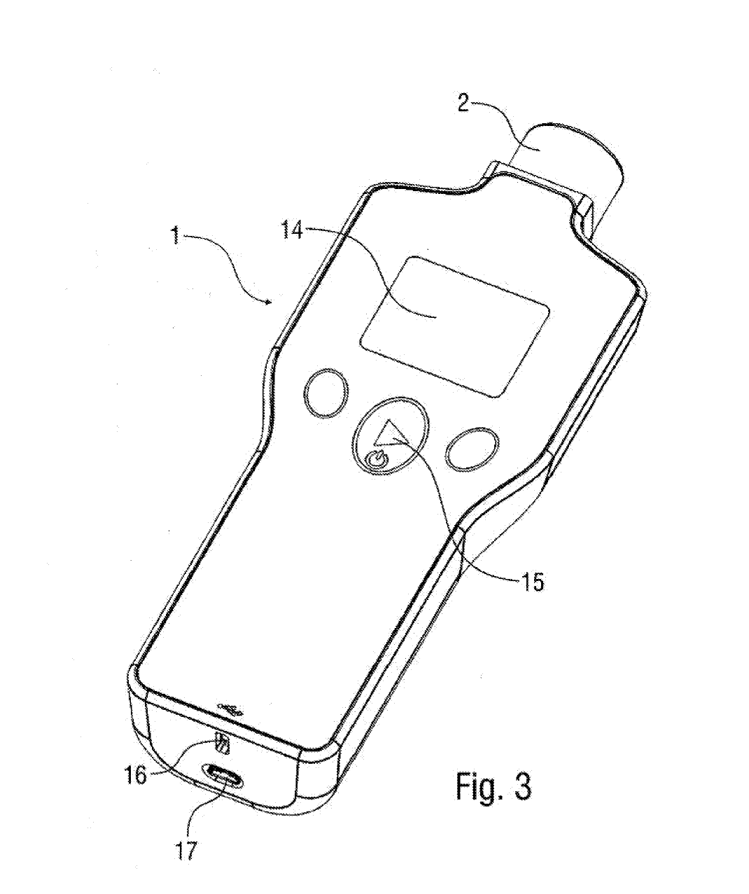

FIG. 3 is an oblique perspective back view of the measuring device of FIGS. 1 and 2,

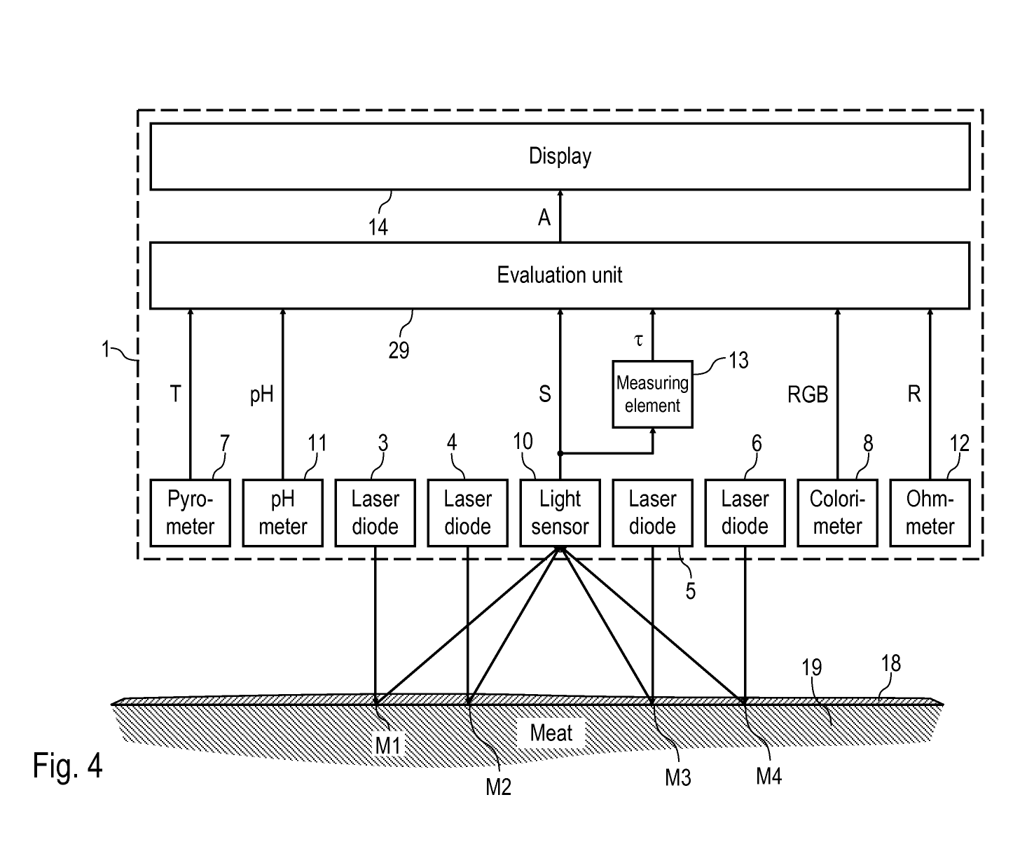

FIG. 4 is a schematic representation of the measuring device according to the invention,

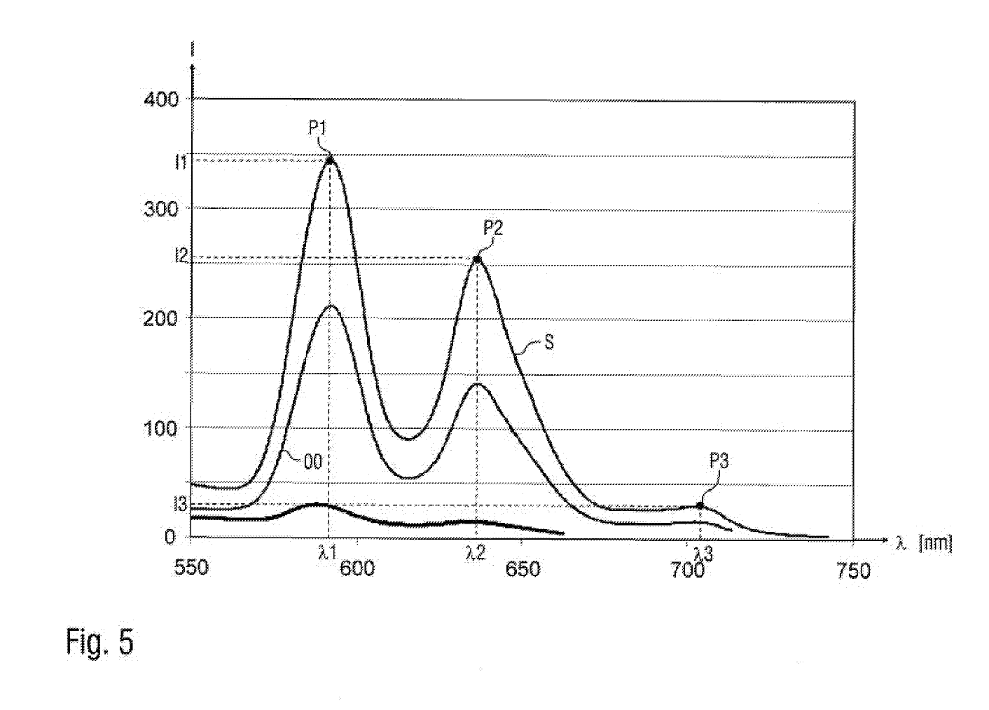

FIG. 5 is a spectral diagram with the spectra of the fluorescent radiation at different times during storage of the meat,

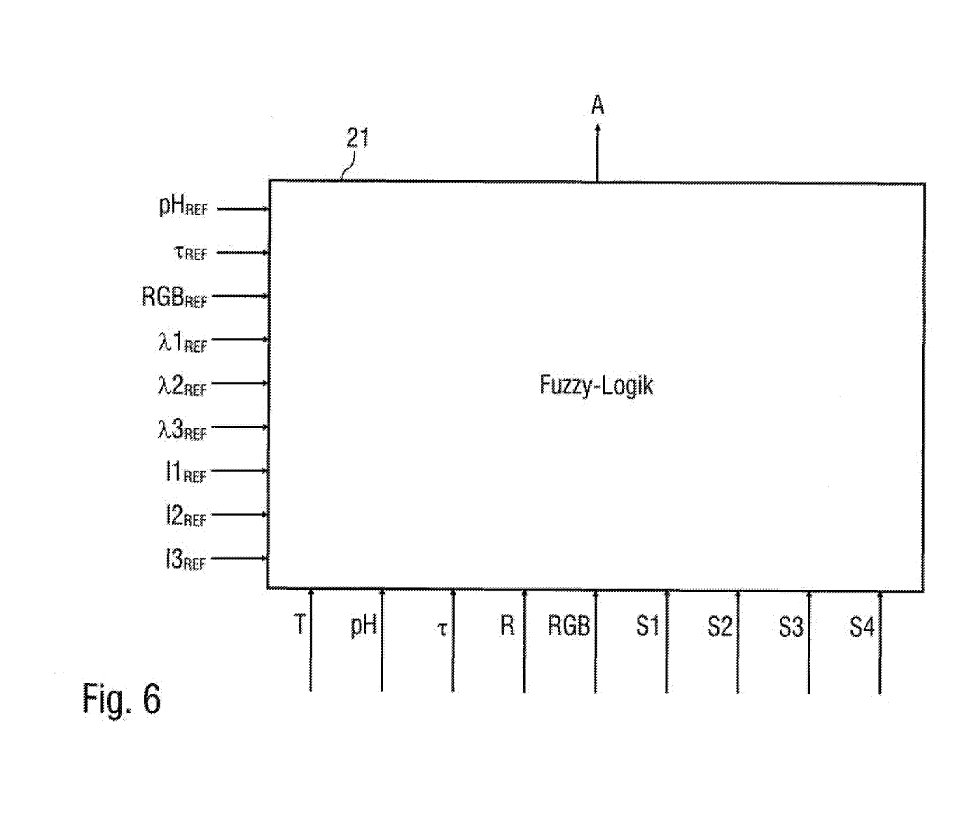

FIG. 6 is a simplified representation for determining the output signal by a fuzzy logic, and

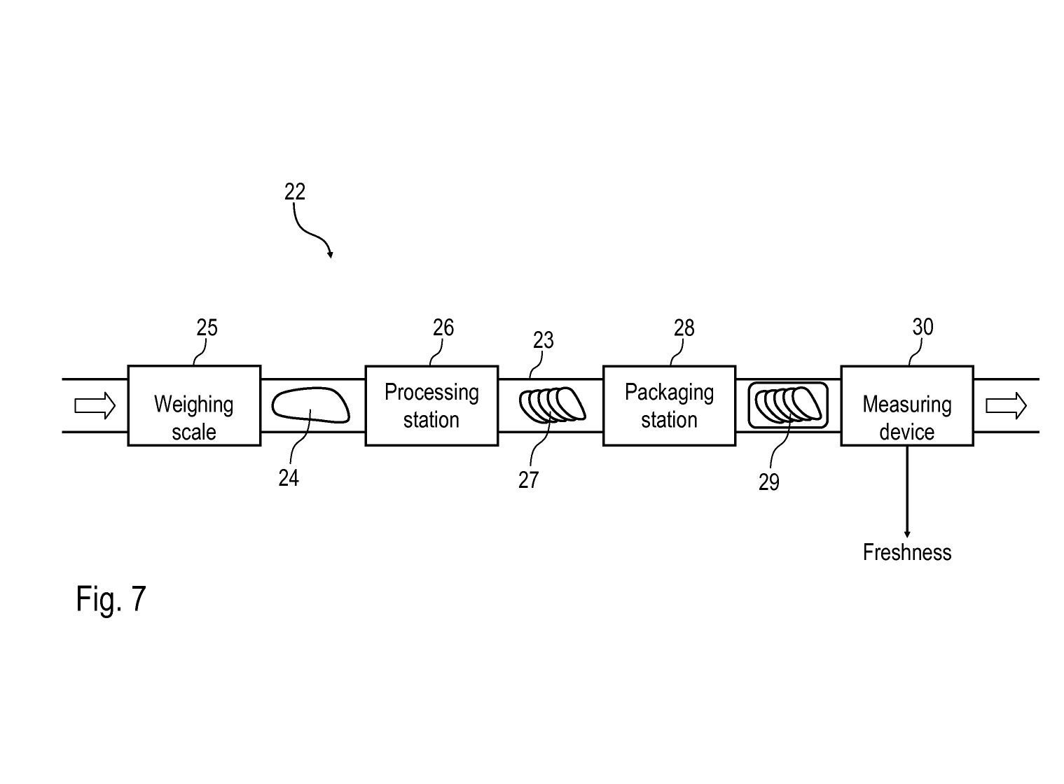

FIG. 7 is a simplified representation of a production line in the food processing industry with the measuring device according to the invention for determining the freshness of the processed meat.

FIGS. 1 to 4 show a measuring device 1 according to the invention for measuring the freshness of meat by exciting and measuring fluorescent radiation emitted by bacterial metabolites (porphyrins) on the meat.

To that end, the measuring device 1 has a measuring head 2 of V4A steel, wherein a transparent measuring cap can be fitted to the measuring head 2 in order to avoid contamination by the foodstuff. Excitation of the fluorescent radiation in the surface layer on the meat and measurement of the fluorescent radiation emitted by the surface layer on the meat are carried out through the transparent measuring cap.

The measuring cap can also have an integrated spectral optical pH indicator microdot. Accordingly, the pH value can also be determined optically via the spectrum.

The measuring head 2 comprises four laser diodes 3-6 which emit ultraviolet light for exciting the fluorescent radiation.

The measuring head 2 further comprises a pyrometer 7 for contactless measurement of the surface temperature of the surface layer on the meat.

The measuring head 2 additionally also comprises a colorimeter 8 having a calibrated light-emitting diode for colorimetric spectral measurement, that is to say for measuring the color of the surface layer without the excitation of fluorescence.

Finally, the measuring head 2 also comprises a collector 9 of an optical fiber, the collector 9 detecting the fluorescent radiation emitted by the surface layer on the meat and transmitting it via the optical fiber to a corresponding optical sensor 10.

The four laser diodes 3-6 illuminate the surface of the meat at four spatially separate measuring points M1-M4, so that fluorescent radiation is generated at each of the four measuring points M1-M4 and is then detected by the collector 9. Measurement at the four different measuring points M1-M4 has the advantage that local irregularities (e.g. as a result of fat inclusions or bones) can be compensated for in the measurement and therefore do not lead to incorrect measurements.

The measuring device 1 according to the invention can additionally have a pH meter 11 which measures the pH value of the surface layer on the meat.

The measuring device can further have an ohmmeter 12 which measures the electrical surface resistance of the surface layer on the meat.

Finally, the measuring device 1 can also have a measuring element 13 which determines the decay behavior over time of the fluorescent radiation emitted by the surface layer on the meat.

On its outer side, the measuring device 1 according to the invention has a display 14, on which the measurement result inter alia is given.

The measuring device 1 additionally has on its upper side a keypad 15, via which user inputs can be made.

Furthermore, a so-called Kensington lock 16 and a USB interface 17 are also arranged in the housing of the measuring device 1.

The operation of the measuring device 1 according to the invention will be described in the following with reference to FIGS. 4 and 5.

The laser diodes 3-6 each illuminate one of the measuring points M1-M4 on a surface layer 18 of meat 19 to be tested. The bacterial metabolites (porphyrins) contained in the surface layer 18 then generate fluorescent radiation, which is measured by the optical sensor 10 via the collector 9.

The optical sensor 10 then gives a corresponding wavelength spectrum S to an evaluation unit 20.

The evaluation unit 20 then determines the peaks P1, P2 and P3 from the measured spectrum S. The intensity I1, I2 and I3 and the wavelength λ1, λ2 and λ3 is measured for each of the individual peaks P1, P2, P3 of the measured fluorescence spectrum S.

The evaluation unit 20 then calculates the intensity ratios V1=I1/I2, V2=I1/I3 and V3=I2/I3. The intensity ratios V1, V2 and V3 are subsequently used as characteristic parameters for qualification of the measured fluorescence spectrum S.

The evaluation unit 20 further determines the wavelength λ1, λ2 and λ3 for each of the peaks P1, P2, P3 of the measured fluorescence spectrum. For each of the peaks P1, P2, P3, the wavelength difference between the measured wavelength λ1, λ2 and λ3, on the one hand, and given characteristic wavelengths λ1_{REF}, λ2_{REF} and λ3_{REF}, on the other hand, is then calculated, the characteristic wavelengths λ1_{REF}, λ2_{REF} and λ3_{REF} being characteristic for the fluorescent radiation of porphyrins. The wavelength differences Δλ1=λ1−λ1_{REF}, Δλ2=λ2−λ2_{REF} and Δλ3=λ3−λ3_{REF} so determined are subsequently used for the qualification of the fluorescence spectrum S.

In addition, the evaluation unit 20 also takes into consideration a temperature T, which is measured by the pyrometer 7, a surface resistance R, which is measured by the ohmmeter 12, a color value RGB, which is measured by the colorimeter 8, and the decay behavior over time, which is measured by the measuring element 13 and is transmitted to the evaluation unit 20 in the form of a time constant T.

The evaluation unit 20 then gives a corresponding evaluation signal A to the display 14, the output signal A indicating the freshness of the meat.

FIG. 6 shows a fuzzy logic 21 for determining the output signal A in dependence on the input parameters described above. The functional principle of such a fuzzy logic is known per se from the prior art and therefore does not require further description.

Finally, FIG. 7 shows an example of a field of use of the invention in a production line 22 for industrial foodstuffs processing.

The production line 22 comprises a conveyor belt 23 on which meat 24 is transported in the direction indicated by the arrow.

At the entry to the production line 22 there is a weighing scale 25, which weighs the meat 24.

Downstream of the weighing scale 25 in the transport direction there is a processing station 26, which processes the meat 24. In this embodiment, the processing station 26 is a cutting device which cuts the meat 24 into a plurality of slices 27.

Downstream of the processing station 26 in the transport direction there is a packaging station 28, which packages the meat slices 27 into a transparent packaging 29.

Downstream of the packaging station 28 in the transport direction there is then a measuring device 30 according to the invention, which measures the freshness of the packaged meat through the transparent packaging 29, as has already been described above.

The invention is not limited to the preferred embodiments described above. Rather, a plurality of variants and modifications are possible which likewise make use of the inventive concept and therefore fall within the scope of protection. In particular, the invention also claims protection for the subject-matter and the features of the dependent claims independently of the claims on which they are dependent. For example, the dependent claims also enjoy protection without the characterizing feature of the main claim.

### LIST OF REFERENCE NUMERALS

A Output signal of the evaluation unit
M1-M4 Measuring points
S Fluorescence spectrum
P1 Peak of the fluorescence spectrum
P2 Peak of the fluorescence spectrum
P3 Peak of the fluorescence spectrum
V1 Intensity ratio I1/I2
V2 Intensity ratio I1/I3
V3 Intensity ratio I2/I3
λ1 Wavelength of peak P1
λ2 Wavelength of peak P2
λ3 Wavelength of peak P3
T Temperature
R Surface resistance
RGB Color value
pH pH value
τ Time constant of the decay behavior of the fluorescent radiation
1 Measuring device
2 Measuring head
3 Laser diode
4 Laser diode
5 Laser diode
6 Laser diode
7 Pyrometer
8 Colorimeter
9 Collector
10 Optical sensor
11 pH meter
12 Ohmmeter
13 Measuring element
14 Display
15 Keypad
16 Kensington lock
17 USB interface
18 Surface layer
19 Meat
20 Evaluation unit
21 Fuzzy logic
22 Production line
23 Conveyor belt
24 Meat
25 Weighing scale
26 Processing station
27 Slices
28 Packaging station
29 Transparent packaging
30 Measuring device

1-14. (canceled)

15. A measuring device for measuring a surface layer on an object to be measured, comprising:
a) at least one excitation source for exciting luminescence in the surface layer on the object to be measured that is to be monitored, so that the surface layer emits luminescent radiation, and
b) at least one optical sensor for detecting the luminescent radiation which is emitted by the surface layer, and
c) an evaluation unit for qualifying and/or quantifying at least one of the surface layer and the object and for generating a corresponding output signal,
d) wherein the evaluation unit determines at least one of the following at peaks of the luminescent radiation:
d1) a wavelength of each peak and a wavelength correspondence between, on the one hand, wavelengths of the peaks of the luminescent radiation and, on the other hand, given characteristic wavelengths, wherein the characteristic wavelengths are wavelengths of peaks in a fluorescent radiation of porphyrins, and
d2) an intensity of each peak and an intensity ratio of the peaks of the luminescent radiation.

16. The measuring device according to claim 15, wherein the measuring device measures the luminescent radiation of the surface layer at a plurality of measuring points on the object to be measured that is to be monitored which are spaced apart from one another.

17. The measuring device according to claim 15, wherein
a) the excitation source is a light source and the luminescent radiation is photoluminescent radiation, and
b) the light source emits light having a spectrum of from 350 nm to 550 nm for exciting photoluminescence, and
c) the light source as the excitation source is a laser diode or a light-emitting diode.

18. The measuring device according to claim 16, wherein the at least one optical sensor detects the luminescent radiation of all the measuring points.

19. The measuring device according to claim 18, wherein the at least one optical sensor has a sufficiently large measuring angle that all the measuring points lie within the measuring angle, so that the at least one optical sensor can measure the luminescent radiation of all the measuring points.

20. The measuring device according to claim 18, wherein the luminescent radiation emitted by the surface layer is guided to the at least one optical sensor via a light guide.

21. The measuring device according to claim 15, wherein the at least one optical sensor is a spectral photometer which measures a wavelength spectrum of the luminescent radiation of the surface layer.

22. The measuring device according to claim 16, wherein the measuring points are distributed around an optical axis of the at least one optical sensor.

23. The measuring device according to claim 15, further comprising a pyrometer for contactless measurement of a surface temperature of the surface layer on the object to be measured that is to be monitored.

24. The measuring device according to claim 15, further comprising a colorimeter for measuring a color of the surface layer without the excitation of luminescence.

25. The measuring device according to claim 15, further comprising a measuring element for measuring a decay behavior over time of the luminescent radiation of the surface layer.

26. The measuring device according to claim 15, further comprising a pH meter for measuring a pH value of the surface layer.

27. The measuring device according to claim 15, further comprising a resistance measuring device for measuring an electrical surface resistance of the surface layer and/or of the object to be measured.

28. The measuring device according to claim 15, wherein the object is a foodstuff and the output signal indicates at least one of the following properties of the foodstuff:
a) bacterial count of metabolites of bacteria in the surface layer,
b) type of foodstuff selected from the group consisting of meat, fish, vegetable and fruit;
c) type of meat selected from the group consisting of pork, beef, poultry, lamb, venison, horsemeat and dog meat, and
d) edibility of the foodstuff in dependence on a freshness of the foodstuff.

29. The measuring device according to claim 15, wherein the evaluation unit qualifies and/or quantifies the surface layer in dependence on at least one of the following parameters:
a) a total value which indicates the intensities of the luminescent radiation at all the measuring points,
b) the intensities of the luminescent radiation at individual measuring points measured by the at least one optical sensor,
c) a surface temperature measured by a pyrometer,
d) a color measured by a colorimeter,
e) a decay behavior over time of the luminescent radiation,
f) an intensity ratio of the peaks of the luminescent radiation,
g) the wavelength correspondence between the measured wavelengths of the peaks of the luminescent radiation and the characteristic wavelengths,
h) a pH value measured by a pH meter, and
i) a surface resistance measured by a resistance measuring device.

30. The measuring device according to claim 15, wherein
a) the measuring device is a hand-held device, and
b) the measuring device contains a battery for supplying power, and
c) the measuring device has an optical display for displaying the output signal.

31. The measuring device according to claim 15, wherein the measuring device has a measuring time which is less than 10 s.

32. The measuring device according to claim 15, wherein the measuring device has at least one data interface for at least one of the following: configuring the measuring device and outputting measured data.

33. The measuring device according to claim 15, wherein the measuring device has a transparent and removable cap, excitation of the luminescent radiation and the measurement of the luminescent radiation taking place through the cap.

34. The measuring device according to claim 16, wherein each measuring point has an associated excitation source so that the excitation of luminescence at each measuring point takes place by the excitation source associated with that measuring point.

35. The measuring device according to claim 15, wherein the measuring device additionally tests the object to be measured by Raman spectroscopy.

36. A method for measuring metabolites of bacteria on a foodstuff that is intended for consumption, said method comprising providing the measuring device according to claim 15 and measuring the metabolites of bacteria on the foodstuff.

37. The method according to claim 36, wherein
a) the foodstuff is processed on a production line, a plurality of processing stations being arranged one after the other along the production line, which processing stations receive, cut, portion, weigh, measure, prepare, plate up and/or package the foodstuff, and
b) the metabolites of bacteria on the foodstuff are measured on the production line by the measuring device.
